(19) United States
(12) Patent Application Publication
(10) Pub. No.: US 2003/0232989 A1
(43) Pub. Date: Dec. 18, 2003

(54) Process for the preparation of specific aromatic aldehydes

(76) Inventors: Stefan Antons, Leverkusen (DE); Joachim Rehse, Leichlingen (DE); Herbert Diehl, Leverkusen (DE); Christian Laue, Monheim (DE)

Correspondence Address: BAYER CHEMICALS CORPORATION, 100 BAYER ROAD, PITTSBURGH, PA, 15205, US

(21) Appl. No.: 10/413,199

(22) Filed: Apr. 14, 2003

(30) Foreign Application Priority Data
Apr. 16, 2002 (DE) 10216967.5

(51) Int. Cl. C07D 215/14; C07D 213/46; C07D 311/96; C07D 239/02; C07C 45/40

(52) U.S. Cl. 544/332; 546/315; 546/168; 548/494; 549/331; 568/430; 562/459; 562/470

(57) ABSTRACT

A new process for the preparation of specific aromatic aldehydes by ozonolysis of aromatic alkenes is provided. This new process can be advantageously integrated into a synthesis of specific chiral diols using aromatic aldehydes of this type.

### BACKGROUND OF THE INVENTION

The invention relates to a process for the preparation of specific aromatic aldehydes by ozonolysis of corresponding aromatic alkenes, and the use of aromatic aldehydes of this type in a process for the preparation of compounds comprising specific chiral diols.

Such compounds of the general formula (VIa)

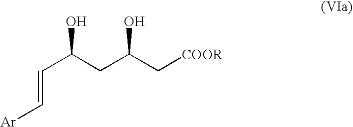

which are described more fully hereinafter are employed in medicaments as HMG-CoA reductase inhibitors for the treatment of lipoproteinaemia on account of their property of inhibiting cholesterol biosynthesis. The following are known examples thereof:

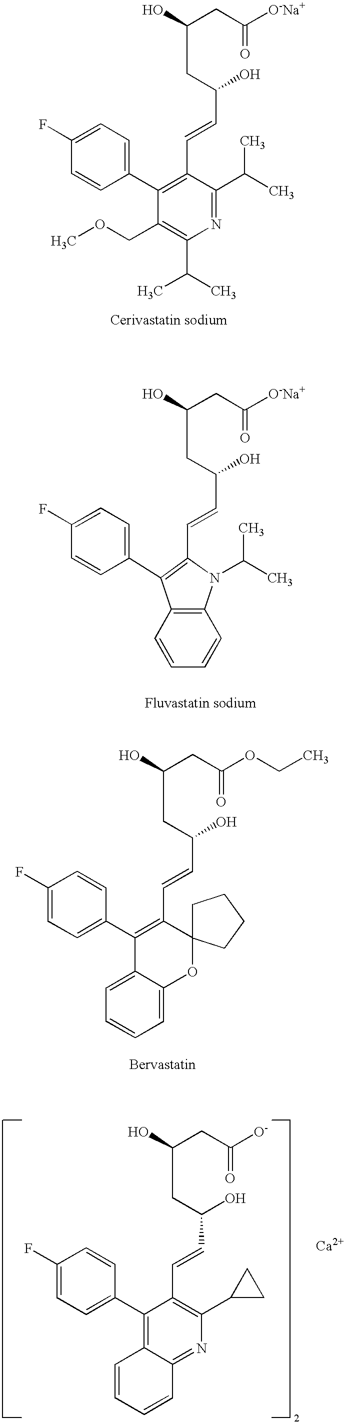

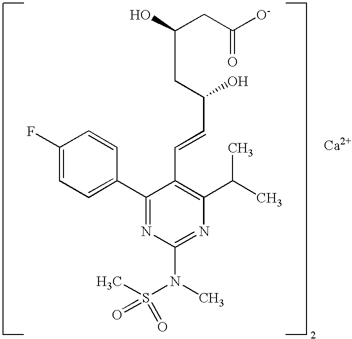

In the compounds of the general formula (VIa), the chiral side chain is customarily constructed by the following synthesis sequence:

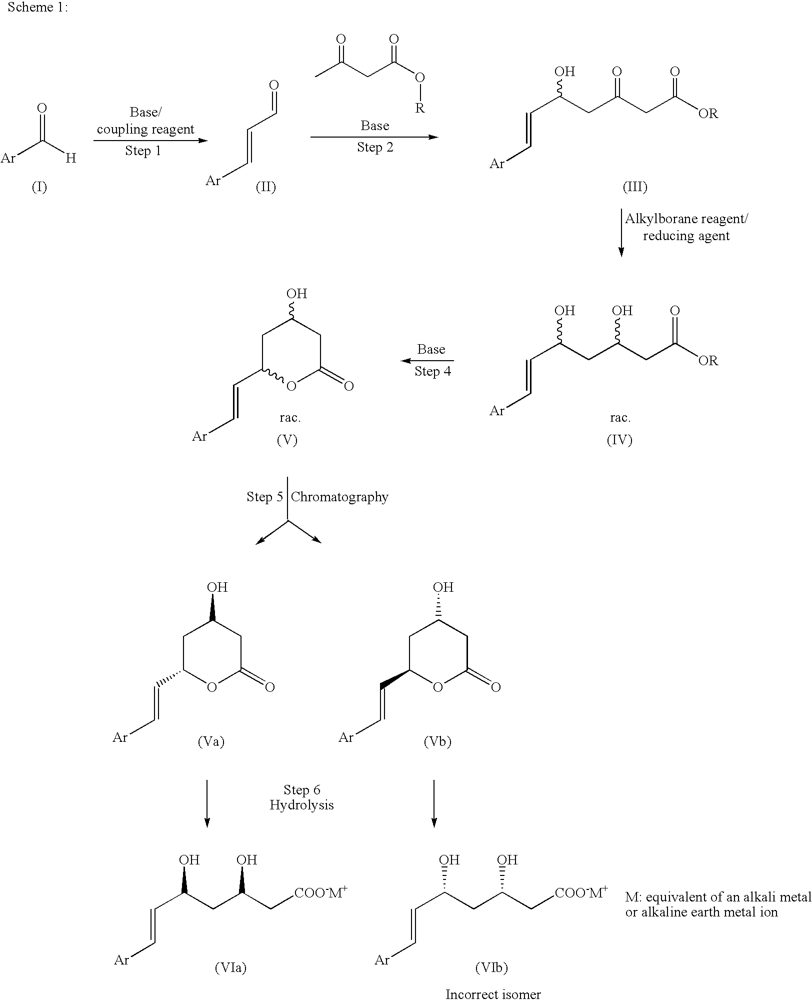

In this scheme, Ar represents a substituted or unsubstituted aryl or heteroaryl radical.

The synthesis of steps 1-3 according to Scheme 1 is disclosed in EP-A-0 603 699, EP-A-0 325 130 and DE-A-40 40 026. Steps 4 and 5 are disclosed in EP-A-0 617 019. Step 4 comprises a conversion of the racemic esters of the general formula (IV) into the racemic lactones of the general formula (V). In Step 5, this racemate is separated into the individual enantiomeric lactones by chromatography on a chiral phase and the enantiomeric lactones are in each case converted into the enantiomerically pure final products (VIa) and (VIb) by hydrolysis.

Owing to the resolution which has to be carried out in Step 5, the overall synthesis is associated with the disadvantage that the overall yield is considerably reduced by the incorrect isomer of the general formula (VIb) which is formed. The economic attractiveness of the overall synthesis is markedly reduced by this, as the incorrect isomer has to be incinerated.

The object of the present invention therefore consisted in putting the incorrect isomer to a further use.

### SUMMARY OF THE INVENTION

The present invention relates to a process for the preparation of aromatic aldehydes of the formula (I)

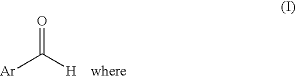

Ar represents a substituted or unsubstituted aryl or heteroaryl radical,

by reaction of compounds of the general formula (VIb) or (Vb)

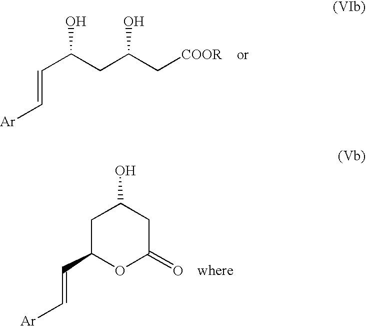

Ar has the meaning mentioned for the general formula (I) and

R denotes hydrogen, straight chain or branched C₁-C₆-alkyl, C₃-C₆-cycloalkyl or an alkali metal or alkaline earth metal ion including equivalents thereof, with ozone.

In the compounds of the general formulae (Vb) and (VIb), Ar represents a substituted or unsubstituted aryl or heteroaryl radical.

### DETAILED DESCRIPTION OF THE INVENTION

The invention is described more fully hereunder with particular reference to its specific embodiments. Ar represents, for example, a pyridyl, pyrimidyl, pyridazine, pyrazine, pyrrole, triazole, quinoline, indole, chromene or chromane radical, which can be identically or differently mono- or polysubstituted. As substituents, further aryl, alkyl, cycloalkyl, halogenoalkyl, halogenoaryl, O—, N— and S-alkyl radicals are possible.

In the compounds of the general formulae (Vb) and (Vib), R preferably represents methyl, ethyl, n-propyl, iso-propyl, n-butyl, iso-butyl or tert-butyl.

In a particularly preferred embodiment, the process according to the invention is carried out using compounds of the formula (Vb) or (VIb), in which Ar represents a radical of the formula

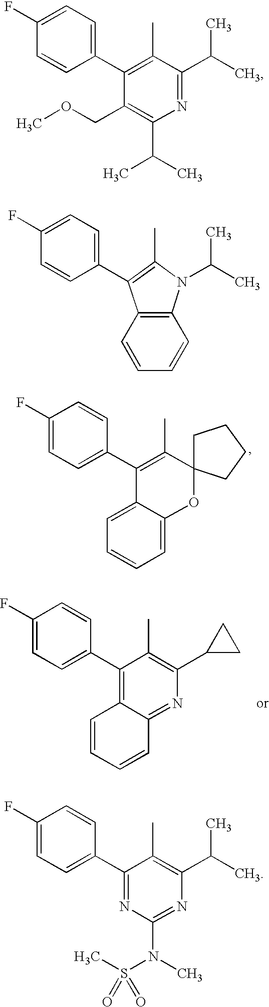

The ozonolysis of the compounds of the general formula (Vb) or (Vib) is customarily carried out in one or more inert polar solvents. Those suitable are, for example, alcohols, in particular methanol or ethanol, organic carboxylic acids, preferably organic C₁-C₆-carboxylic acids, in particular formic acid or acetic acid, aldehydes, preferably C₃-C₆-aldehydes, in particular formaldehyde or acetaldehyde, ketones, preferably acetone, methyl ethyl ketone, methyl isobutyl ketone or cyclohexanone.

The aromatic alkene of the formula (Vb) or (VIb) is introduced in the inert polar solvent and the ozone is passed into this solution. The temperature of the reaction mixture in this process is in the range from −100 to +30° C., preferably in the range from −40 to +20° C. Rates of addition of 0.01 to 10 mol of ozone/mole of aromatic alkene per hour, preferably of 0.1 to 1 mol of ozone per mole of aromatic alkene per hour, have proved suitable. Customarily, ozone and the aromatic alkene of the formula (Vb) or (VIb) are employed in total in a molar ratio of (1:1)-(1:10), preferably (1:1)-(1:2).

After completion of the introduction of ozone the reaction mixture is worked up. The following procedure has proved suitable here: excess ozone which may be present is destroyed using a sulfur compound such as dimethyl sulfide, an alkali metal sulfide or alkali metal hydrogensulfide. After removal of the solvent, the mixture is purified either by distillation, chromatography or crystallization.

More conveniently, by means of the ozonolysis according to the invention the aromatic aldehyde of the formula (I) is obtained, which is the starting material for the synthesis of the chiral side chain according to Scheme 1. Recycling of the aromatic aldehyde obtained by the ozonolysis according to the invention into the overall synthesis according to Scheme I is thus possible. By this means, the economic attractiveness of the overall process increases very considerably.

The invention therefore also relates to a process for the preparation of compounds of the general formula (VIa) by

(1) reaction of an aldehyde of the general formula (I)

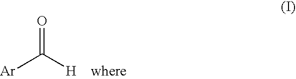

Ar represents a substituted or unsubstituted aryl or heteroaryl radical,

with a phosphonate ester or triphenylphosphonium salt as a coupling reagent in the presence of an organic or inorganic base with formation of a compound of the general formula (II),

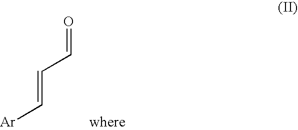

Ar has the meaning mentioned for the formula (I),

(2) reaction of the compound of the general formula (II) with an acetoacetic ester of a straight-chain or branched C₁-C₆-alcohol in the presence of a base with formation of a compound of the general formula (III)

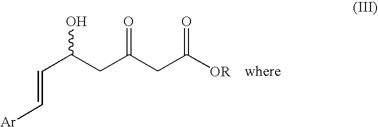

Ar has the meaning mentioned for the formula (I) and

R represents a straight chain or branched C₁-C₆-alkyl radical,

(3) reaction of the compound of the general formula (III) with an alkylborane reagent in the presence of a reducing agent with formation of a racemate of the general formula (IV)

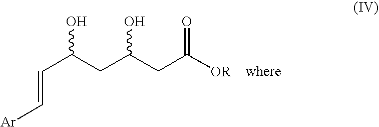

Ar has the meaning mentioned for the formula (I) and

R has the meaning mentioned for the formula (III),

(4) reaction of the racemate of the general formula (IV) with a base and cyclization with formation of a racemate of the general formula (V)

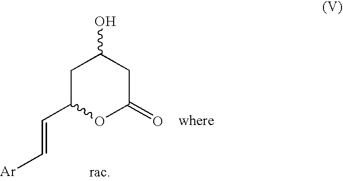

Ar has the meaning mentioned for the formula (I),

(5) chromatographic resolution of the racemate of the general formula (V) into the enantiomeric lactones of the general formulae (Va) and (Vb)

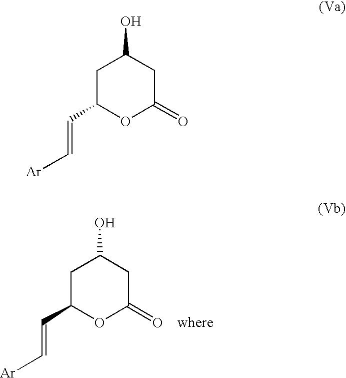

Ar has the meaning mentioned for the formula (I),

(6) separate hydrolysis of these enantiomeric lactones of the general formulae (Va) and (Vb) with formation of the compounds of the general formulae (VIa) and (VIb)

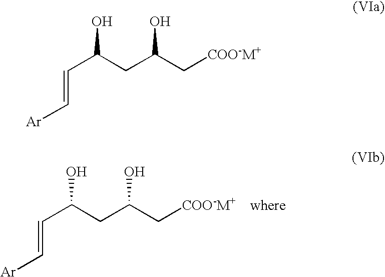

Ar has the meaning mentioned for the formula (I), and

M represents the equivalent of an alkali metal or alkaline earth metal,

(7) reaction of a compound of the general formula (Vb) or (VIb)

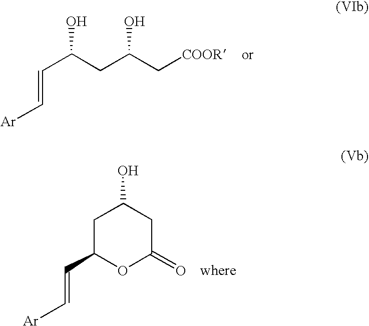

Ar has the meaning mentioned for the formula (I) and

R′ denotes hydrogen, straight chain or branched C₁-C₆-alkyl, C₃-C₆-cycloalkyl or an alkali metal or alkaline earth metal ion including equivalents thereof,

with ozone with formation of the aldehyde of the general formula (I) and

(8) recycling of this aldehyde of the general formula (I) into Step (1).

In Step 1, the aldehyde is firstly reacted with a phosphonate ester or triphenyl-phosphonium salt as a coupling reagent in the presence of an organic or inorganic base. Phosphonate esters are particularly preferred as a coupling reagent.

Inorganic bases which can be employed are alkali metal hydrides, hydroxides, carbonates or hydrogencarbonates. Organic bases which have proved suitable are trialkylamines or pyridine derivatives.

The treatment of the aldehyde is in general carried out in a temperature range from −100 to +100° C., preferably from −20 to +100° C., particularly preferably from 0 to +50° C.

Suitable solvents during the treatment with the base are ethers such as, for example, diethyl ether, dioxane or tetrahydrofuran or alternatively mixtures thereof.

The acrolein of the general formula (II) formed is liberated from the reaction mixture by addition of an acid, preferably of an inorganic acid. Purification is preferably carried out by crystallization or distillation.

In Step 2, the acrolein of the general formula (II) obtained is reacted with an acetoacetic ester of a straight chain or branched C₁-C₆-alcohol in the presence of a base, preferably of a strong base. NaOH and butyllithium are particularly preferred. Alternatively, the sodium salt of an acetoacetic ester can also be employed. The treatment of the acrolein of the general formula (II) is in general carried out in a temperature range from −100 to +100° C., preferably from −50 to +20° C., particularly preferably from −30 to +20° C.

Suitable solvents during the treatment with the base are ethers such as, for example, diethyl ether, dioxane or tetrahydrofuran. Tetrahydrofuran is particularly preferably employed.

During this reaction, a compound of the general formula (III) is formed, which is a racemic hydroxyketoester. The liberation of the compound of the general formula (III) from the reaction mixture is carried out by addition of an acid, preferably of an inorganic acid. Purification is preferably carried out by crystallization or distillation.

In Step 3, the hydroxyketoester of the general formula (III) obtained in Step 2 is reacted with an alkylborane reagent in the presence of a reducing agent to give a compound of the general formula (IV), which is a racemic dihydroxyester.

Suitable reducing agents are complex metal hydrides, such as, for example, lithium aluminum hydride, sodium cyanoborohydride, sodium aluminum hydride, diisobutylaluminum hydride, sodium borohydride or sodium bis-(2-methoxy-ethoxy)dihydroaluminate.

The treatment of the hydroxyketoester in Step 3 is in general carried out in a temperature range from −100 to 0° C., preferably from −80 to −21° C. and particularly preferably from −78 to −50° C.

Suitable solvents are ethers such as, for example, diethyl ether, dioxane or tetrahydrofuran. Tetrahydrofuran is particularly preferably employed.

The racemic dihydroxyester of the general formula (IV) formed is liberated by addition of an acid, preferably of an inorganic acid. Purification is preferably carried out by crystallization or distillation.

In Step 4, the racemic dihydroxyesters of the general formula (IV) are converted into the racemic lactones of the formula (V).

This conversion is in general carried out by treating the ester with bases and subsequent cyclization in suitable solvents with elimination of water. In this reaction, the carboxylic acids and their salts are formed intermediately.

Suitable bases for this are customary inorganic bases such as sodium hydroxide, potassium hydroxide, sodium carbonate or potassium carbonate. Sodium hydroxide is preferably employed.

Suitable solvents during the treatment with the base are ethers such as, for example, diethyl ether, dioxane or tetrahydrofuran. Tetrahydrofuran is particularly preferably employed.

Subsequent cyclization by heating is in general carried out in solvents which are inert under the reaction conditions. These include hydrocarbons such as benzene, toluene, xylene, petroleum fractions, tetralin, diglyme or triglyme. Benzene, toluene and xylene are particularly preferably employed. It is also possible to employ mixtures of the solvents mentioned. Toluene is particularly preferably used. The water eliminated in this reaction can be removed by azeotropic distillation or by means of molecular sieve. Azeotropic distillation is particularly preferred.

The treatment of the esters of the general formula (IV) with bases is in general carried out in a temperature range from 0-50° C., preferably from 10-30° C., particularly preferably at 20° C.

The cyclization is in general carried out in a temperature range from 0 to +200° C., preferably from +25 to +150° C. It is preferably carried out at the boiling temperature of the azeotropic mixture of the solvent used in each case with water.

In Step 5, the enantiomers of the racemate (V) are then separated into the individual enantiomeric lactones by chromatography on a chiral phase and the enantiomeric lactones are then converted into the enantiomerically pure final products or their alkali metal or alkaline earth metal salts by hydrolysis.

The chromatographic separation of the racemic lactones into the individual enantiomerically pure lactones is in general carried out on customary chiral materials. These preferably include optically active polymers of optically active (meth)acrylic acid derivatives. Polymers of optically active N-(meth)acryloyl-amino acid derivatives are particularly preferred here, such as are described in EP-A-0 379 917. Polymers of the following optically active N-acryloyl-amino acid esters may be very particularly preferably mentioned here: N-acryloyl-L- or D-amino acid menthyl esters, a suitable amino acid being, for example, leucine, alanine, phenylalanine, valine or other amino acids.

Mobile phases used for the separation of the racemate are customary organic solvents or solvent mixtures which swell the polymer employed as an adsorbent and dissolve the racemate to be used. The following may be mentioned by way of example: hydrocarbons such as benzene, toluene or xylene, ethers such as diethyl ether, dioxane or tetrahydrofuran, halogenohydrocarbons such as di- or trichloro-methane, acetone, acetonitrile or ethyl acetate or else mixtures of the solvents mentioned. Mixtures of toluene and tetrahydrofuran and of toluene and dioxane have proved particularly suitable.

The hydrolysis of the respective enantiomerically pure lactone to the desired enantiomerically pure final product according to Step 6 is carried out in a customary manner using a base in organic solvents.

Possible solvents here are the customary organic solvents which are inert under the reaction conditions. Ethers such as diethyl ether, dioxane or tetrahydrofuran may preferably be mentioned here. Tetrahydrofuran is particularly preferably employed.

Suitable bases are the customary inorganic bases such as alkali metal hydroxides or alkali metal carbonates. Sodium hydroxide and potassium hydroxide are preferred.

The hydrolysis is in general carried out in a temperature range from 0-60° C., preferably from 10 to 50° C., particularly preferably at 20° C.

After carrying out Steps 2-4 of the process according to the invention, reaction mixtures are in each case obtained which, in addition to the respective products of the individual step, i.e. the compounds of the general formulae (III), (IV) and (V), also contain still unreacted starting materials, i.e. compounds of the general formulae (II), (III) and/or (IV). The reaction mixture which remains after separating off the desired products (“mother liquor”) can likewise be subjected to an ozonolysis, the aldehyde of the general formula (I) likewise being obtained.

The present invention thus also relates to a process for the preparation of aromatic aldehydes of the formula (I)

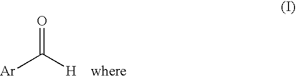

Ar represents a substituted or unsubstituted aryl or heteroaryl radical,

by reaction of a reaction mixture containing one more of the compounds of the general formula (VII)

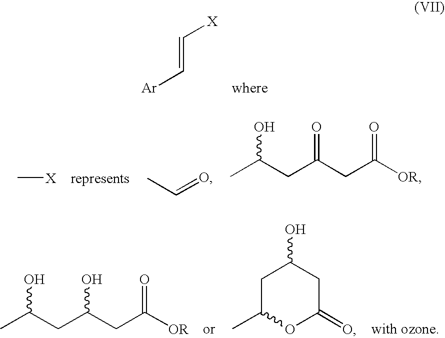

The conditions for an ozonolysis of the mother liquors of this type can be chosen analogously to the conditions mentioned for the ozonolysis of the compounds of the formula (Vb) or (VIb). This additional ozonolysis of one or more of the mother liquors from Steps 2 to 4 of the process according to be invention can be carried out individually or in combined form, instead of or else additionally to the ozonolysis of the compounds of the general formula (Vb) or (VIb). The total yield of the process is further increased thereby.

The invention thus also relates to a process for the preparation of compounds of the general formula (VIa) by

(1) reaction of an aldehyde of the general formula (I)

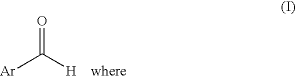

Ar represents a substituted or unsubstituted aryl or heteroaryl radical,

with a phosphonate ester or triphenylphosphonium salt as a coupling reagent in the presence of an organic or inorganic base with formation of a compound of the general formula (II),

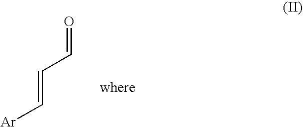

Ar has the meaning mentioned for the formula (I),

(2) reaction of the compound of the general formula (II) with an acetoacetic ester of a straight-chain or branched C₁-C₆-alcohol in the presence of a base with formation of a compound of the general formula (III)

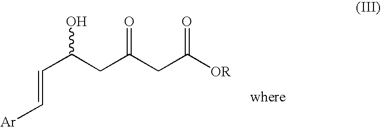

Ar has the meaning mentioned for the formula (I) and

R represents a straight chain or branched C₁-C₆-alkyl radical,

(3) reaction of the compound of the general formula (III) with an alkylborane reagent in the presence of a reducing agent with formation of a racemate of the general formula (IV)

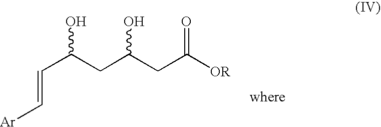

Ar has the meaning mentioned for the formula (I) and

R has the meaning mentioned for the formula (III),

(4) reaction of the racemate of the general formula (IV) with a base and cyclization with formation of a racemate of the general formula (V)

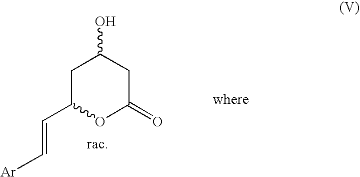

Ar has the meaning mentioned for the formula (I),

(5) chromatographic resolution of the racemate of the general formula (V) into the enantiomeric lactones of the general formulae (Va) and (Vb)

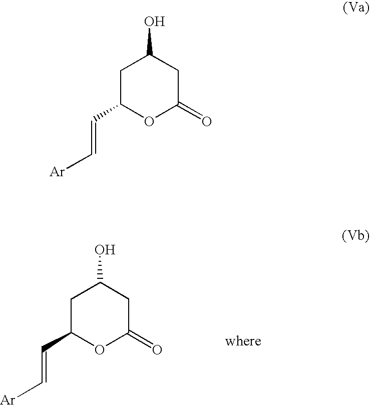

Ar has the meaning mentioned for the formula (I),

(6) separate hydrolysis of these enantiomeric lactones of the general formulae (Va) and (Vb) with formation of the compounds of the general formulae (VIa) and (VIb)

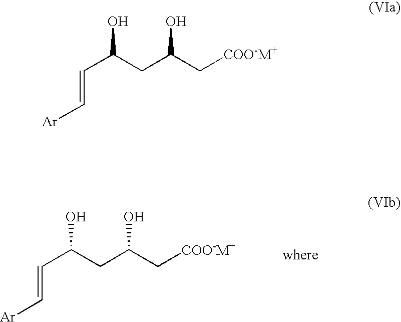

Ar has the meaning mentioned for the formula (I), and

M represents the equivalent of an alkali metal or alkaline earth metal,

(7) reaction of one or more of the reaction mixtures which are obtained according to Steps (2), (3) and/or (4) after separation of the compounds of the formulae (III), (IV) and (V), containing one or more of the compounds of the general formula (VII)

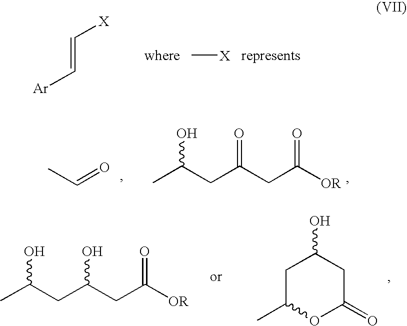

individually or in combined form with ozone with formation of the aldehyde of the general formula (I) and

(8) recycling of this aldehyde of the general formula (I) into Step (1).

The invention is described further by way of the following illustrative but non-limiting examples.

### EXAMPLES

### Example 1

Ozonolysis of (E)-6-{2-(2,6-diisopropyl-4-(4-fluorophenyl)-3-methoxymethyl-pyrid-5-yl)-ethenyl)-3,4,5,6-tetrahydro-4-hydroxy-2H-pyran-2-one (1) to 2,6-diisopropyl-4-(4-fluorophenyl)-5-methoxymethyl-pyridine-3-carbaldehyde (2)

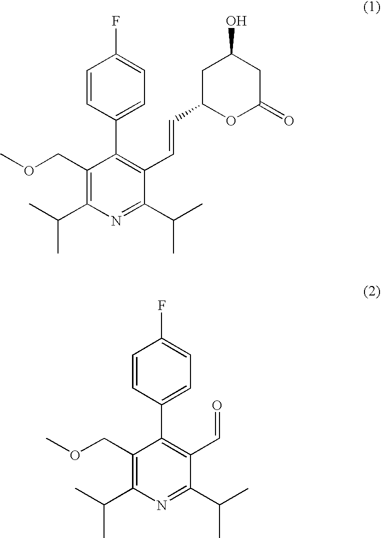

Ozone is passed with stirring into a solution of 4.0 g (9.1 mmol) of the aromatic alkene (1) in 160 ml of methanol p.a. cooled to −40° C. An ozone generator from Fischer is used here. The rate of addition of ozone is 301 of O₂/h and about 2 g of O₃/h. After 17 min (corresponding to 11 mmol of O₃), the ozonolysis is terminated, as a slight blue coloration of the reaction mixture has taken place. The reaction mixture is flushed with nitrogen at −40° C. and treated with 600 mg (9.67 mmol) of dimethyl sulfide at this temperature. The reaction mixture is allowed to warm to 20° C. in the course of 30 min and the mixture is allowed to stand for a further 2.5 h. The solvent is then removed by distillation and the residue is dissolved in a little methylene chloride, treated with 10 g of silica gel and freed of the methylene chloride. The silica gel loaded with the desired product is shaken with 100 g of silica gel on a frit and eluted with 12, 50 ml-fractions of cyclohexane-ethyl acetate (volume ratio 20:1).

The product fractions contain 2.5 g (83%) of a colorless oil, which crystallizes in substance.

### Example 2

Ozonolysis of (E)-6-{2-(2,6-diisopropyl-4-phenyl-3-methoxymethyl-pyrid-5-yl)-ethenyl)-3,4,5,6-tetrahydro-4-hydroxy-2H-pyran-2-one (3) to 2,6-diisopropyl-4-phenyl-5-methoxymethyl-pyridine-3-carbaldehyde (4)

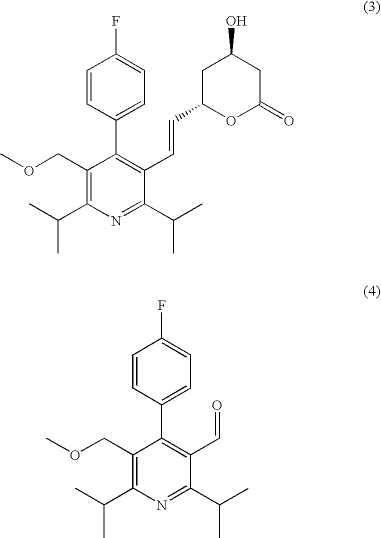

From the overall process according to Scheme 1, the mother liquor of the chromatography in Step 5, which consists of 90% of the incorrect isomer (Vb), is freed as far as possible of the solvent on a rotary evaporator under a reduced pressure of about 20 mbar at a bath temperature of about 40° C.

The dark-brown oily residue which remains is dissolved in isopropanol (10 g in 75 ml) and treated with 75 ml of 50% strength acetic acid at 20° C. The solution is cooled to −20 to −30° C. Ozone is then passed in with stirring for a period of 1 h. An ozone generator from Sander is used. The ozone is passed in at a rate of addition of about 351 of O₂/h and about 2 g of O₃/h. O₂ is then passed through the reaction solution for a further hour without an ozone generator.

The mixture is treated with about 10 ml of dimethyl sulfide at −30° C. and the reaction mixture is allowed to warm to 20° C. with stirring over the course of 12 hours. If the iodide/starch test for ozonides is negative, the reaction mixture is freed of the solvent under reduced pressure. If the iodide/starch test is positive, the reaction mixture is treated again with dimethyl sulfide until the test is negative.

The residue which remains after concentration is dissolved in 250 ml of ethyl acetate, washed with 100 ml of saturated NaHCO₃ solution, 100 ml of saturated NaCl solution and 100 ml of water and then dried over Na₂SO₄. The residue which remains after the removal of the solvent is recrystallized from isopropanol/H₂O 1:1.

Yield: about 5 g of the aldehyde (4) corresponding to 90% of theory

Purity: about 97% (HPLC)

### Example 3

Ozonolysis of Mother Liquors from the Reaction of the Aldehyde (4) According to Scheme 1

The mother liquors obtained after Steps 1, 2 and 3 in a reaction of aldehyde (4) according to Scheme 1 are combined and freed as far as possible of the solvent under a reduced pressure of about 20 mbar at a bath temperature of about 40° C. The dark-brown oily residue which remains is dissolved in isopropanol (10 g in 75 ml) and treated with 75 ml of 50% strength acetic acid at 20° C. The solution is cooled to −20 to −30° C.

Ozone is then passed in for about 1 h with stirring. An ozone generator from Sander is used. The ozone is passed in at a rate of addition of about 351 of O₂/h, and about 2 g of O₃/h. O₂ is then passed through the reaction solution for a further hour without an ozone generator. The mixture is treated at −30° C. with about 10 ml of dimethyl sulfide and the reaction mixture is allowed to warm to 20° C. over the course of 12 hours with stirring.

If the iodide/starch test for ozonides is negative, the solvent is largely removed under reduced pressure. If the iodide/starch test is positive, the mixture is again treated with dimethyl sulfide until the test is negative.

The residue which remains after extensive removal of the solvent is dissolved in 250 ml of ethyl acetate, washed with 100 ml of saturated NaHCO₃ solution, 100 ml of saturated NaCl solution and 100 ml of water and then dried over Na₂SO₄. The residue which remains after removal of the solvent is recrystallized from isopropanol/H₂O 1:1.

Yield: about 5 g of the aldehyde (4) corresponding to 70% of theory

Purity: about 97% (HPLC)

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for the preparation of aromatic aldehydes of the formula (I) 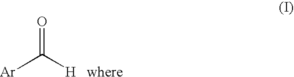
Ar represents a substituted or unsubstituted aryl or heteroaryl radical,
comprising reacting compounds of the general formula (VIb) or (Vb) 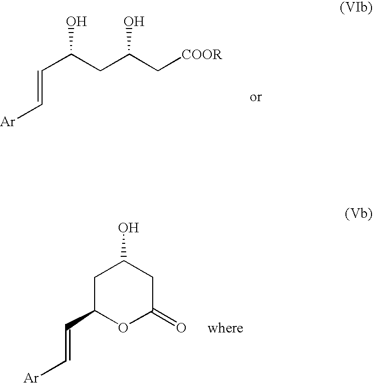
Ar has the meaning mentioned for the general formula (I) and
R denotes hydrogen, straight chain or branched C₁-C₆-alkyl, C₃-C₆-cycloalkyl or an alkali metal or alkaline earth metal ion,
with ozone.

2. Process according to claim 1, characterized in that compounds of the general formulae (Vb) and (VIb) are employed, in which R represents methyl, ethyl, n-propyl, iso-propyl, n-butyl, iso-butyl or tert-butyl.

3. Process according to claim 1, characterized in that compounds of the general formulae (VIb) and (Vb) are employed, in which Ar represents

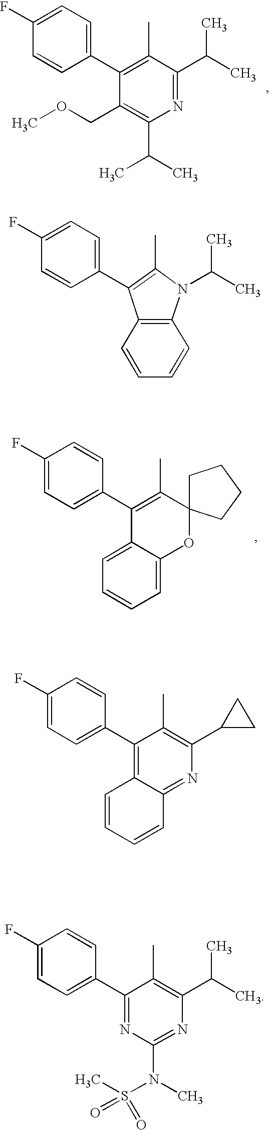

4. Process according to claim 1, characterized in that the reaction with ozone is carried out in one or more inert polar solvents.

5. Process according to claim 1, characterized in that the reaction with ozone is carried out at a temperature of the reaction mixture in the range from −100 to +30° C.

6. Process according to claim 1, characterized in that the reaction with ozone is carried out at a rate of addition of 0.01-10 mol of ozone per mole of aromatic alkene per hour.

7. Process according to claim 1, characterized in that ozone and the aromatic alkene of the general formula (Vb) or (VIb) are employed 1 in a molar ratio of (1:1)-(1:10).

8. Process for the preparation of compounds of the general formula (VIa) 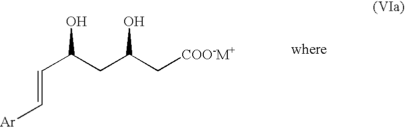
Ar represents a substituted or unsubstituted aryl or heteroaryl radical, and
M represents an alkali metal or alkaline earth metal ion,
comprising
(1) reacting an aldehyde of the general formula (I) 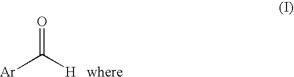
Ar represents a substituted or unsubstituted aryl or heteroaryl radical,
with a phosphonate ester or triphenylphosphonium salt as a coupling reagent in the presence of an organic or inorganic base with formation of a compound of the general formula (II), 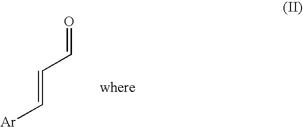
Ar has the meaning mentioned for the formula (I),
(2) reacting the compound of the general formula (II) with an aceto-acetic ester of a straight-chain or branched C₁-C₆-alcohol in the presence of a base with formation of a compound of the general formula (III) 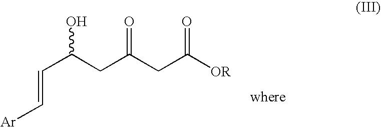
Ar has the meaning mentioned for the formula (I) and
R represents a straight chain or branched C₁-C₆-alkyl radical,
(3) reacting the compound of the general formula (III) with an alkyl-borane reagent in the presence of a reducing agent with formation of a racemate of the general formula (IV) 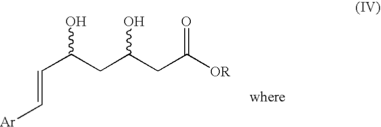
Ar has the meaning mentioned for the formula (I) and
R has the meaning mentioned for the formula (III),
(4) reacting the racemate of the general formula (IV) with a base and cyclization with formation of a racemate of the general formula (V) 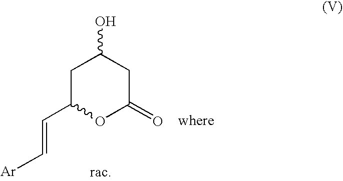
Ar has the meaning mentioned for the formula (I),
(5) conducting chromatographic resolution of the racemate of the general formula (V) into the enantiomeric lactones of the general formulae (Va) and (Vb) 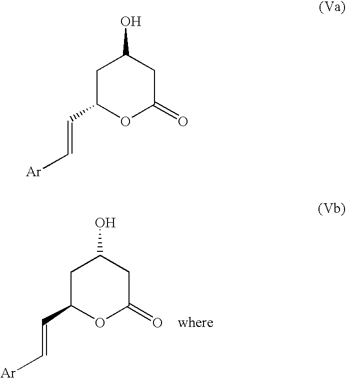
Ar has the meaning mentioned for the formula (I),
(6) hydrolyzing separately these enantiomeric lactones of the general formulae (Va) and (Vb) with formation of the compounds of the general formulae (VIa) and (VIb) 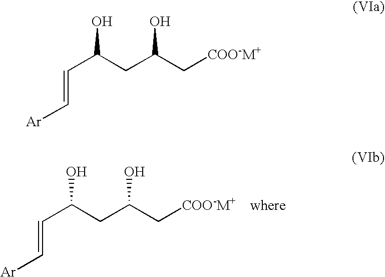
Ar has the meaning mentioned for the formula (I), and
M represents the equivalent of an alkali metal or alkaline earth metal,
(7) reacting a compound of the general formula (Vb) or (VIb) 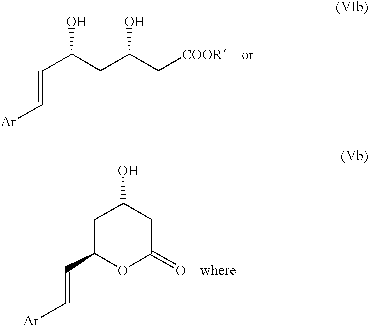
Ar has the meanings mentioned for the formula (I) and
R′ denotes hydrogen, straight chain or branched C₁-C₆-alkyl, C₃-C₆-cycloalkyl or an alkali metal or alkaline earth metal ion,
with ozone with formation of the aldehyde of the general formula (I) and
(8) recycling of this aldehyde of the general formula (I) into Step (1).

9. Process for the preparation of aromatic aldehydes of the formula (I) 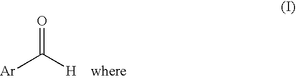
Ar represents a substituted or unsubstituted aryl or heteroaryl radical,
comprising reacting a reaction mixture containing one or more of the compounds of the general formula (VII) 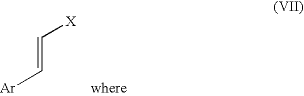
Ar has the meaning mentioned for the general formula (I), and

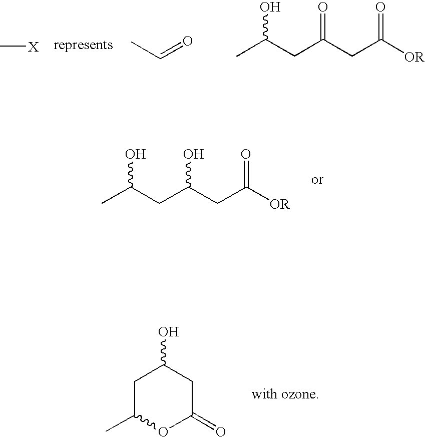

10. Process for the preparation of compounds of the general formula (VIa) 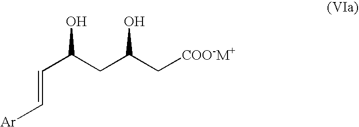
Ar represents a substituted or unsubstituted aryl or heteroaryl radical, and
M represents an alkali metal or alkaline earth metal ion, by
(1) reacting an aldehyde of the general formula (I) 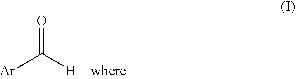
Ar represents a substituted or unsubstituted aryl or heteroaryl radical,
with a phosphonate ester or triphenylphosphonium salt as a coupling reagent in the presence of an organic or inorganic base with formation of a compound of the general formula (II), 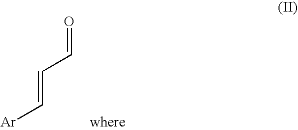
Ar has the meaning mentioned for the formula (I),
(2) reacting the compound of the general formula (II) with an acetoacetic ester of a straight-chain or branched C₁-C₆-alcohol in the presence of a base with formation of a compound of the general formula (III) 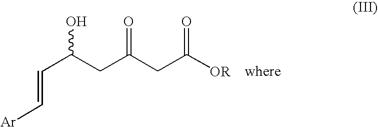
Ar has the meaning mentioned for the formula (I) and
R represents a straight chain or branched C₁-C₆-alkyl radical,
(3) reacting the compound of the general formula (III) with an alkyl-borane reagent in the presence of a reducing agent with formation of a racemate of the general formula (IV) 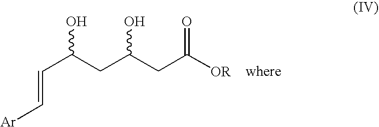
Ar has the meaning mentioned for the formula (I) and
R has the meaning mentioned for the formula (III),
(4) reacting the racemate of the general formula (IV) with a base and cyclization with formation of a racemate of the general formula (V) 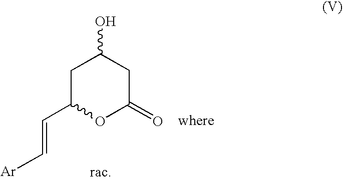
Ar has the meaning mentioned for the formula (I),
(5) conducting chromatographic resolution of the racemate of the general formula (V) into the enantiomeric lactones of the general formulae (Va) and (Vb) 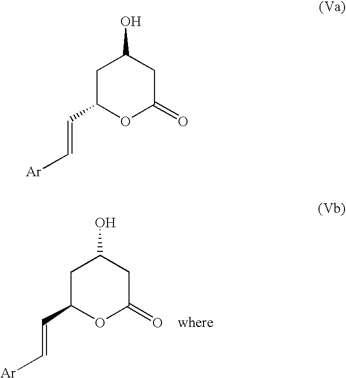
Ar has the meaning mentioned for the formula (I),
(6) hydrolyzing separately these enantiomeric lactones of the general formulae (Va) and (Vb) with formation of the compounds of the general formulae (VIa) and (VIb) 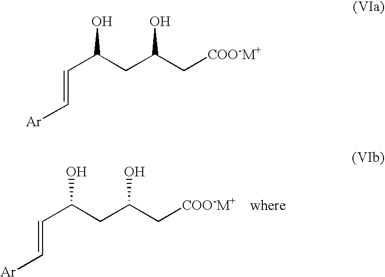
Ar has the meaning mentioned for the formula (I), and
M represents the equivalent of an alkali metal or alkaline earth metal,
(7) reacting one or more of the reaction mixtures which are obtained according to Steps (2), (3) and/or (4) after separation of the compounds of the formulae (III), (IV) and (V), containing one or more of the compounds of the general formula (VII) 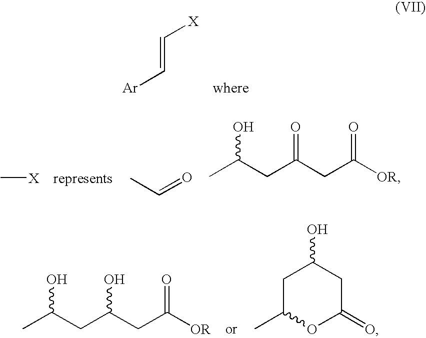
individually or in combined form with ozone with formation of the aldehyde of the general formula (I) and
(8) recycling of this aldehyde of the general formula (I) into Step (1).
